(19) United States
(12) Patent Application Publication
(10) Pub. No.: US 2025/0186453 A1
(43) Pub. Date: Jun. 12, 2025

(54) COMBINATION THERAPIES COMPRISING A SOS1 INHIBITOR AND A MEK INHIBITOR

(71) Applicant: Mirati Therapeutics, Inc., San Diego, CA (US)

(72) Inventors: Jacob Haling, San Diego, CA (US); Shilpi Khare, San Diego, CA (US)

(21) Appl. No.: 18/852,225

(22) PCT Filed: Apr. 3, 2023
(86) PCT No.: PCT/US2023/017254

§ 371 Date: Sep. 27, 2024

Related U.S. Application Data
(60) Provisional application No. 63/329,104, filed on Apr. 8, 2022.

(51) Int. Cl. A61K 31/5377; A61K 31/166; A61K 31/4184; A61K 31/4523; A61K 31/506; A61K 31/519; A61P 35/00

(52) U.S. Cl. CPC A61K 31/5377; A61K 31/166; A61K 31/4184; A61K 31/4523; A61K 31/506; A61K 31/519; A61P 35/00

(57) ABSTRACT

The present invention relates to combination therapies for treating cancers associated with genetic alterations of the MAPK pathway and/or EGFR. In particular, the present invention relates to methods of treating cancer in a subject in need thereof, comprising administering to the subject a therapeutically effective amount of a combination of a SOS1 inhibitor and MEK inhibitor, pharmaceutical compositions comprising a such compositions, kits comprising such compositions and methods of use therefor.

### FIELD OF THE INVENTION

The present invention relates to combination therapies useful for treating cancer. In particular, the present invention relates to therapeutically effective combinations of a Son of sevenless homolog 1 (SOS1) inhibitor and a mitogen-activated protein kinase kinase (MEK) inhibitor, pharmaceutical compositions comprising the inhibitors, kits comprising the compositions and methods of use therefor.

### BACKGROUND OF THE INVENTION

### SOS1 Inhibitors

The Ras family comprises v-Ki-ras2 Kirsten rat sarcoma viral oncogene homolog (KRas), neuroblastoma RAS viral oncogene homolog (NRAS), and Harvey murine sarcoma virus oncogene (HRas) and critically regulates cellular division, growth and function in normal and altered states including cancer (see e.g., Simanshu et al. Cell, 2017. 170(1): p. 17-33; Matikas et al., Crit Rev Oncol Hematol, 2017. 110: p. 1-12). RAS proteins are activated by upstream signals, including receptor tyrosine kinases (RTKs), and transduce signals to several downstream signaling pathways such as the mitogen-activated protein kinase (MAPK)/extracellular signal-regulated kinases (ERK) pathway. Hyperactivation of RAS signaling is frequently observed in cancer as a result of mutations or alterations in RAS genes or other genes in the RAS pathway. The identification of strategies to inhibit RAS and RAS signaling are predicted to be useful for the treatment of cancer and RAS-regulated disease states.

RAS proteins are guanosine triphosphatases (GTPases) that cycle between an inactive, guanosine diphosphate (GDP)-bound state and an active guanosine triphosphate (GTP)-bound state. RAS proteins exhibit both intrinsic GTP hydrolysis and nucleotide exchange, which is further enhanced by extrinsic GTPase activating proteins (GAPs) and guanine echange factors (GEFs). Son of sevenless homolog 1 (SOS1) is a GEF that mediates the exchange of GDP for GTP, thereby activating RAS proteins. This regulation through GAPs and GEFs is the mechanism whereby activation and deactivation are tightly regulated under normal conditions. Mutations at several residues in all three RAS proteins are frequently observed in cancer and result in RAS remaining predominantly in the activated state (Sanchez-Vega et al., Cell, 2018. 173: p. 321-337 Li et al., Nature Reviews Cancer, 2018. 18: p. 767-777). Mutations at codon 12 and 13 disrupt the GTP hydrolysis and exchange rate of RAS proteinse t. Recent biochemical analysesdemonstrated these mutated proteins still require nucleotide cycling for activation based on their intrinsic GTPase activity and may exhibit partial sensitivity to extrinsic GAPs and GEFs. As such, mutant RAS proteins are sensitive to inhibition of upstream factors such as the SOS1 GEF (Hillig, 2019; Patricelli, 2016; Lito, 2016; Nichols, 2018).

The three main RAS-GEF families that have been identified in mammalian cells are SOS, RAS-GRF and RAS-GRP (Rojas, 2011). RAS-GRF and RAS-GRP are expressed in the cells of the central nervous system and hematopoietic cells, respectively, while the SOS family is ubiquitously expressed and is responsible for transducing RTK signaling. The SOS family comprises SOS1 and SOS2 and these proteins share approximately 70% sequence identity. SOS1 appears to be much more active than SOS2 due to the rapid degradation of SOS2. The mouse SOS2 knockout is viable whereas the SOS1 knockout is embryonic lethal. A tamoxifen-inducible SOS1 knockout mouse model was used to interrogate the role of SOS1 and SOS2 in adult mice and demonstrated the SOS1 knockout was viable but the SOS1/2 double knockout was not viable (Baltanas, 2013) suggesting functional redundancy and that selective inhibition of SOS1 may have a sufficient therapeutic index for the treatment of SOS1-RAS activated diseases.

SOS proteins are recruited to phosphorylated RTKs through an interaction with growth factor receptor bound protein 2 (GRB2). Recruitment to the plasma membrane places SOS in close proximity to RAS and enables SOS-mediated RAS activation. SOS proteins bind to RAS through a catalytic binding site that promotes nucleotide exchange as well as through an allosteric site that binds GTP-bound RAS-family proteins which increases the catalytic function of SOS (Freedman et al., Proc. Natl. Acad. Sci, USA 2006. 103(45): p. 16692-97). Binding to the allosteric site relieves steric occlusion of the catalytic site and is therefore required for full activation of the catalytic site. Retention of the active conformation at the catalytic site following interaction with the allosteric site is maintained in isolation due to strengthened interactions of key domains in the activated state. SOS1 mutations are found in Noonan syndrome and several cancers including lung adenocarcinoma, embryonal rhabdomyosarcoma, Sertoli cell testis tumor and granular cell tumors of the skin (see e.g., Denayer, E., et al, Genes Chromosomes Cancer, 2010. 49(3): p. 242-52).

GTPase-activating proteins (GAPs) are proteins that stimulate the low intrinsic GTPase activity of RAS family members and therefore converts active GTP-bound RAS proteins into inactive, GDP-bound RAS proteins (e.g., see Simanshu, D. K., Cell, 2017, Ras Proteins and their Regulators in Human Disease). While activating alterations in the phosphatase PTPN11(SHP2) and the GEF SOS1 occur in cancers, inactivating mutations and loss-of-function alterations in the GAP neurofibromin 1 (NF-1) also occur creating a state where SOS1 activity is unopposed and activity downstream of the pathway through RAS proteins is elevated.

### MEK Inhibitors

The mitogen-activated protein kinase (MAPK) signaling pathway is involved in the regulation of various cellular activities, including, but not limited to cell proliferation, survival, differentiation, and motility. The classical MAPK pathway consists of Ras (a family of related proteins which is expressed in all animal cell lineages and organs), Raf (a family of three serine/threonine-specific protein kinases that are related to retroviral oncogenes), MEK (mitogen-activated protein kinase kinase), and ERK (extracellular signal-regulated kinases), sequentially relaying proliferative signals generated at the cell surface receptors into the nucleus through cytoplasmic signaling.

MEK inhibitors target the Ras/Raf/MEK/ERK signaling pathway, thereby inhibiting cell proliferation and inducing apoptosis.

The MAPK pathway is one of the most commonly mutated oncogenic pathways in cancer, Deregulation of this pathway is frequently observed and plays a central role in the carcinogenesis and maintenance of several cancers, including melanoma, pancreatic, lung, colorectal, and breast cancers. (e.g., Neuzillet et al., (2014) Pharmacology & Therapeutics 141:160-171).

Several inhibitors exhibiting activity against MEK have been developed, and a number of these inhibitors are or have been inventigated in human clinical trials. Examples of MEK inhibitors suitable for the provided compositions and methods include, but are not limited to selumetinib, 6-(4-bromo-2-chloroanilino)-7-fluoro-N-(2-hydroxyethoxy)-3-methylbenzimidazole-5-carboxamide; AZD8330, 2-(2-fluoro-4-iodoanilino)-N-(2-hydroxyethoxy)-1,5-dimethyl-6-oxopyridine-3-carboxamide; PD0325901, N-[(2R)-2,3-dihydroxypropoxy]-3,4-difluoro-2-(2-fluoro-4-iodoanilino)benzamide; PD318088, 5-bromo-N-(2,3-dihydroxypropoxy)-3,4-difluoro-2-(2-fluoro-4-iodoanilino)benzamide; refametinib, N-[3,4-difluoro-2-(2-fluoro-4-iodoanilino)-6-methoxyphenyl]-1-[(2S)-2,3-dihydroxypropyl]cyclopropane-1-sulfonamide; binimetinib, 6-(4-bromo-2-fluoroanilino)-7-fluoro-N-(2-hydroxyethoxy)-3-methylbenzimidazole-5-carboxamide; RO4987655, 3,4-difluoro-2-(2-fluoro-4-iodoanilino)-N-(2-hydroxyethoxy)-5-[(3-oxooxazinan-2-yl)methyl]benzamide, RO5126766 (VS-6766), 3-[[3-fluoro-2-(methylsulfamoylamino)pyridin-4-yl]methyl]-4-methyl-7-pyrimidin-2-yloxychromen-2-one; WX-554, HL-085; ClnQ-03; G-573, 7-fluoro-3-(2-fluoro-4-iodoanilino)-N-[(2S)-2-hydroxypropoxy]furo[3,2-c]pyridine-2-carboxamide; PD184161, 5-bromo-2-(2-chloro-4-iodoanilino)-N-(cyclopropylmethoxy)-3,4-difluorobenzamide; RO5068760, (2S,3S)-2-[(4R)-4-[4-[(2R)-2,3-dihydroxypropoxy]phenyl]-2,5-dioxoimidazolidin-1-yl]-N-(2-fluoro-4-iodophenyl)-3-phenylbutanamide; SL327, (Z)-3-amino-3-(4-aminophenyl)sulfanyl-2-[2-(trifluoromethyl)phenyl]prop-2-enenitrile; MEK162 (Arry-162), 5-((4-bromo-2-fluorophenyl)amino)-4-fluoro-N-(2-hydroxyethoxy)-1-methyl-1H-benzo[d]imidazole-6-carboxamide; Tak-733, (R)-3-(2,3-dihydroxypropyl)-6-fluoro-5-((2-fluoro-4-iodophenyl)amino)-8-methylpyrido[2,3-d]pyrimidine-4,7(3H,8H)-dione; GDC-0623, 5-((2-fluoro-4-iodophenyl)amino)-N-(2-hydroxyethoxy)imidazo[1,5-a]pyridine-6-carboxamide; U0126, (2E,3E)-2-(amino((2-aminophenyl)thio)methylene)-3-(amino((3-aminophenyl)thio)methylene)succinonitrile; trametinib, N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide; BI-847325, (E)-3-(3-(((4-((dimethylamino)methyl)phenyl)amino)(phenyl)methylene)-2-oxoindolin-6-yl)-N-ethylpropiolamide; pimasertib, (S)—N-(2,3-dihydroxypropyl)-3-((2-fluoro-4-iodophenyl)amino)isonicotinamide; BIX02189, (Z)-3-(((3-((dimethylamino)methyl)phenyl)amino)(phenyl)methylene)-N,N-dimethyl-2-oxoindoline-6-carboxamide; cobimetinib, (3,4-difluoro-2-((2-fluoro-4-iodophenyl)amino)phenyl)(3-hydroxy-3-(piperidin-2-yl)azetidin-1-yl)methanone; PD-98059, 2-(2-amino-3-methoxyphenyl)-4H-chromen-4-one; APS-2-79, 6,7-dimethoxy-N-(2-methyl-4-phenoxyphenyl)quinazolin-4-amine; PD 198306, N-(cyclopropylmethoxy)-3,4,5-trifluoro-2-(4-iodo-2-methylbenzyl)benzamide; CI-1040, 2-(2-chloro-4-iodobenzyl)-N-(cyclopropylmethoxy)-3,4-difluorobenzamide; SL327, (Z)-3-amino-3-((4-aminophenyl)thio)-2-(2-(trifluoromethyl)phenyl)acrylonitrile; and BIX02188, (Z)-3-(((3-((dimethylamino)methyl)phenyl)amino)(phenyl)methylene)-2-oxoindoline-6-carboxamide.

Structures of these MEK inhibitors can be found, for example, in Cheng et al, Current Development Status of MEK Inhibitors, Molecules 2017, 22, 1551, as well as in U.S. Pat. Nos. 10,370,374 and 10,323,035, as well as in US Patent Application Publication Nos: 20190144382; 20180370948; 20180296533; 20180147192; 20180118715; 20170231963; 20170183348; 20170183333; 20170166523; 20170101408; 20170096388; 20160331753; 20160168103; 20160168102; 20160136150; 20160108041; 20150141399; 20150133424; 20150051209; 20140378466; 20140275527; 20140135519; 20140128442; 20140080804; 20130150573; 20130018075; 20120238599; 20120208859; 20120107307; 20120022076; 20110288092; 20110263558; 20110190257; 20110183981; 20110172191; 20110158971; 20110124622; 20110112152; 20110060049; 20110021558; 20100331334; 20100267710; 20100261718; 20100261717; 20100260714; 20100256149; 20100249096; 20100197676; 20100179124; 20100063053; 20090291961; 20090264411; 20090233915; 20090215834; 20090209542; 20090156576; 20090149437; 20090143579; 20090143389; 20090131435; 20090082457; 20090030058; 20080306063; 20080280957; 20080255133; 20080177082; 20080171778; 20080166359; 20080058340; 20070299063; 20070293544; 20070287737; 20070287709; 20070244164; 20070238710; 20070213367; 20070172843; 20070112038; 20070105859; 20060211073; 20060194802; 20060189808; 20060189668; 20060189649; 20060154990; 20060106225; 20060089382; 20060052608; 20050256123; 20050250782 and 20050153942, the contents of which are hereby incorporated by reference in their entirety.

One of the MEK inhibitors suitable for the provided compositions and methods is VS-6766 which has the following structure:

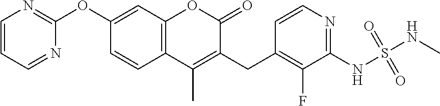 [SMILES: CNS(=O)(=O)Nc1nccc(Cc2c(C)c3ccc(Oc4ncccn4)cc3oc2=O)c1F]

3-[[3-fluoro-2-(methylsulfamoylamino)pyridin-4-yl]methyl]-4-methyl-7-pyrimidin-2-yloxychromen-2-one

Methods for manufacturing MEK inhibitors, or pharmaceutically acceptable salts or pharmaceutical compositions thereof are well known to those skilled in the art and MEK inhibitors may be obtained from a wide-variety of commercial suppliers, in forms suitable for both research or human use. In addition, suitable MEK inhibitors for use in the compositions and methods disclosed herein and methods for preparing such inhibitors are disclosed in US Patent Application Publication Nos: 20190144382; 20180370948; 20180296533; 20180147192; 20180118715; 20170231963; 20170183348; 20170183333; 20170166523; 20170101408; 20170096388; 20160331753; 20160168103; 20160168102; 20160136150; 20160108041; 20150141399; 20150133424; 20150051209; 20140378466; 20140275527; 20140135519; 20140128442; 20140080804; 20130150573; 20130018075; 20120238599; 20120208859; 20120107307; 20120022076; 20110288092; 20110263558; 20110190257; 20110183981; 20110172191; 20110158971; 20110124622; 20110112152; 20110060049; 20110021558; 20100331334; 20100267710; 20100261718; 20100261717; 20100260714; 20100256149; 20100249096; 20100197676; 20100179124; 20100063053; 20090291961; 20090264411; 20090233915; 20090215834; 20090209542; 20090156576; 20090149437; 20090143579; 20090143389; 20090131435; 20090082457; 20090030058; 20080306063; 20080280957; 20080255133; 20080177082; 20080171778; 20080166359; 20080058340; 20070299063; 20070293544; 20070287737; 20070287709; 20070244164; 20070238710; 20070213367; 20070172843; 20070112038; 20070105859; 20060211073; 20060194802; 20060189808; 20060189668; 20060189649; 20060154990; 20060106225; 20060089382; 20060052608; 20050256123; 20050250782 and 20050153942.

### SUMMARY OF THE INVENTION

The combination therapy of the present invention, in one aspect, synergistically increases the potency of SOS1 inhibitors resulting in improved efficacy of SOS1 inhibitors disclosed herein. The combination therapy of the present invention, in another aspect, provides improved clinical benefit to patients compared to treatment with SOS1 inhibitors disclosed herein as a single agent.

Thus in one aspect of the invention there are provided therapeutically effective combinations of a SOS1 inhibitor such as those described in WO2021/127429, WO2021/173524, WO2022/026465, U.S. provisional patent application 63/213,112 (and corresponding national and international applications and publications) and as described in greater detail herein, for instance:

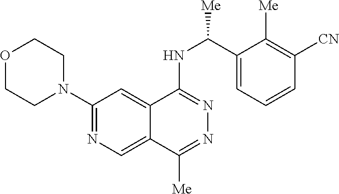 [SMILES: Cc1c(C#N)cccc1[C@@H](C)Nc1nnc(C)c2cnc(N3CCOCC3)cc12]

(R)-2-methyl-3-(1-((4-methyl-7-morpholinopyrido[3,4-cd]pyridazin-1-yl)amino)ethyl)benzonitrile,

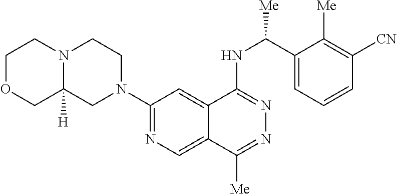 [SMILES: Cc1c(C#N)cccc1[C@@H](C)Nc1nnc(C)c2cnc(N3CCN4CCOC[C@@H]4C3)cc12]

3-((R)-1-((7-((S)-hcxahydropyrazino[2,1-c][1,4]oxazin-8(1H)-yl)-4-mcthylpyrido[3,4-d]pyridazin-1-yl)amino)ethyl)-2-methylbenzonitrile,

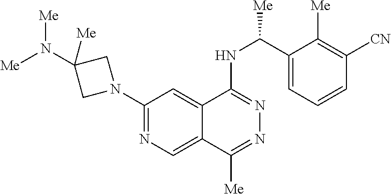 [SMILES: Cc1c(C#N)cccc1[C@@H](C)Nc1nnc(C)c2cnc(N3CC(C)(N(C)C)C3)cc12]

(R)-3-(1-((7-(3-(dimethylamino)-3-methylazetidin-1-yl)-4-methylpyrido[3,4-d]pyridazin-1-yl)amino)ethyl)-2-methylbenzonitrile,

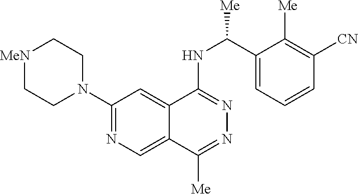 [SMILES: Cc1c(C#N)cccc1[C@@H](C)Nc1nnc(C)c2cnc(N3CCN(C)CC3)cc12]

(R)-2-methyl-3-(1-((4-methyl-7-(4-methylpiperazin-1-yl)pyrido[3,4-d]pyridazin-1-yl)amino)ethyl)benzonitrile,

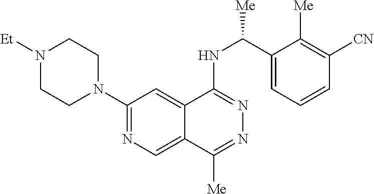 [SMILES: CCN1CCN(c2cc3c(N[C@H](C)c4cccc(C#N)c4C)nnc(C)c3cn2)CC1]

(R)-3-(1-((7-(4-ethylpiperazin-1-yl)-4-methylpyrido[3,4-d]pyridazin-1-yl)amino)ethyl)-2-methylbenzonitrile,

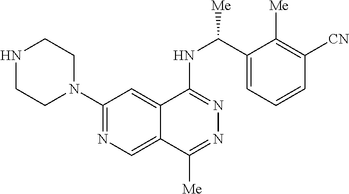 [SMILES: Cc1c(C#N)cccc1[C@@H](C)Nc1nnc(C)c2cnc(N3CCNCC3)cc12]

(R)-2-methyl-3-(1-((4-methyl-7-(piperazin-1-yl)pyrido[3,4-d]pyridazin-1-yl)amino)ethyl)benzonitrile,

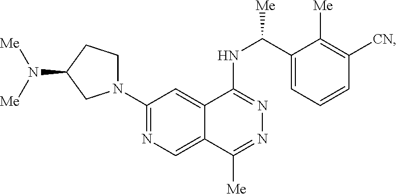 [SMILES: Cc1c(C#N)cccc1[C@@H](C)Nc1nnc(C)c2cnc(N3CC[C@H](N(C)C)C3)cc12]

3-((R)-1-((7-((S)-3-(dimethylamino)pyrrolidin-1-yl)-4-methylpyrido[3,4-d]pyridazin-1-yl)amino)ethyl)-2-methylbenzonitrile,

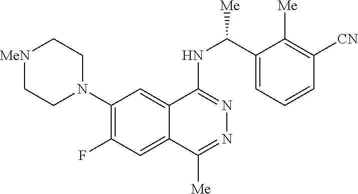 [SMILES: Cc1c(C#N)cccc1[C@@H](C)Nc1nnc(C)c2cc(F)c(N3CCN(C)CC3)cc12]

(R)-3-(1-((6-fluoro-4-methyl-7-(4-methylpiperazin-1-yl)phthalazin-1-yl)amino)ethyl)-2-methylbenzonitrile,
or a pharmaceutically acceptable salt thereof,
and a MEK inhibitor compound such as VS-6766, binimetinib, cobimetinib, selumetinib, trametinib, PD-325901, CI-1040, TAK733, and other small and large molecule MEK inhibitors, or a pharmaceutically acceptable salt thereof.

In another aspect of the invention there are provided therapeutically effective combinations of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and a MEK inhibitor selected from VS-6766, binimetinib, cobimetinib, selumetinib, trametinib, PD-325901, CI-1040, CI-1040 and TAK733 and other small and large molecule MEK inhibitors, or a pharmaceutically acceptable salt thereof.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the MEK inhibitor compound VS-6766, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the MEK inhibitor compound binimetinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the MEK inhibitor compound cobimetinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the MEK inhibitor compound selumetinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the MEK inhibitor compound trametinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the MEK inhibitor compound PD-325901, or a pharmaceutically acceptable salt thereof, and a pharmaceutically acceptable excipient.

In one aspect of the invention, provided herein are methods of treating cancer in a subject in need thereof, comprising administering to the subject a therapeutically effective amount of a combination of a SOS1 inhibitor such as (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino)pyrrolidin-1-yl)-4-methylpyrido[3,4-d]pyridazin-1-yl)amino)ethyl)-2-methylbenzonitrile or (R)-3-(1-((6-fluoro-4-methyl-7-(4-methylpiperazin-1-yl)phthalazin-1-yl)amino)ethyl)-2-methylbenzonitrile, and a MEK inhibitor selected from VS-6766, binimetinib, cobimetinib, selumetinib, trametinib, PD-325901, CI-1040, TAK733, and other small and large molecule MEK inhibitors, or a pharmaceutically acceptable salt or a pharmaceutical composition thereof

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the MEK inhibitor VS-6766, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the MEK inhibitor binimetinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the MEK inhibitor cobimetinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the MEK inhibitor selumetinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the MEK inhibitor trametinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the MEK inhibitor PD-325901, or a pharmaceutically acceptable salt or a pharmaceutical composition thereof.

In one embodiment, the cancer is a SOS1-associated cancer.

In one embodiment, the SOS1-associated cancer is brain cancer, for instance glioblastoma. In one embodiment, the SOS1-associated cancer is lung cancer, for instance primary lung adenocarcinoma. In one embodiment, the SOS1-associated cancer is lung cancer, for instance primary lung adenocarcinoma. In one embodiment, the SOS1-associated cancer is leukemia, for instance acute myeloid leukemia.

In some aspects of the invention, MEK inhibitor(s) and SOS1 inhibitor(s) are the only active agents in the provided compositions and methods.

Examples of SOS1 inhibitors suitable for the provided compositions and methods include, but are not limited to (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino)pyrrolidin-1-yl)-4-methylpyrido[3,4-d]pyridazin-1-yl)amino)ethyl)-2-methylbenzonitrile and (R)-3-(1-((6-fluoro-4-methyl-7-(4-methylpiperazin-1-yl)phthalazin-1-yl)amino)ethyl)-2-methylbenzonitrile, and pharmaceutically acceptable salts thereof. In yet another aspect, the invention provides for methods for increasing the sensitivity of a cancer cell to a SOS1 inhibitor, or to a MEK inhibitor, comprising contacting the cancer cell with a therapeutically effective amount of a combination of a MEK inhibitor compound such as VS-6766, binimetinib, cobimetinib, selumetinib, trametinib, PD-325901, CI-1040, and TAK733, and other small and large molecule MEK inhibitors, or a pharmaceutically acceptable salt or a pharmaceutical composition thereof, and a SOS1 inhibitor or a pharmaceutically acceptable salt or a pharmaceutical composition thereof, including the SOS1 inhibitors described herein such as (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino)pyrrolidin-1-yl)-4-methylpyrido[3,4-d]pyridazin-1-yl)amino)ethyl)-2-methylbenzonitrile and (R)-3-(1-((6-fluoro-4-methyl-7-(4-methylpiperazin-1-yl)phthalazin-1-yl)amino)ethyl)-2-methylbenzonitrile, and pharmaceutically acceptable salts thereof, to synergistically increase the sensitivity of the cancer cell to the SOS1 or MEK inhibitor. In one embodiment, the contacting is in vitro. In one embodiment, the contacting is in vivo.

Also provided herein are methods for treating cancer in a subject in need thereof, the method comprising (a) determining that cancer is associated with a SOS1 mediated cancer and/or genetic alterations of the MAPK pathway (e.g., a SOS1-associated cancer) (e.g., as determined using a regulatory agency-approved, e.g., FDA-approved, assay or kit); and (b) administering to the patient a therapeutically effective amount of a combination of a SOS1 inhibitor, or a pharmaceutically acceptable salt or a pharmaceutical composition thereof, and a MEK inhibitor or a pharmaceutically acceptable salt or a pharmaceutical composition thereof, wherein the MEK inhibitor synergistically increases the sensitivity of the SOS1-associated cancer to the SOS1 inhibitor, for instane to (R)-2-methyl-3-(1-((4-methyl-7-morpholinopyrido[3,4-d]pyridazin-1-yl)amino)ethyl)benzonitrile.

Also provided herein are kits comprising a SOS1 inhibitor, or a pharmaceutically acceptable salt or a pharmaceutical composition thereof and a MEK inhibitor compound or a pharmaceutically acceptable salt or a pharmaceutical composition thereof. Also provided is a kit comprising the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the MEK inhibitor compound VS-6766 or a pharmaceutically acceptable salt or a pharmaceutical composition thereof, for use in treating a SOS1-associated cancer.

In a related aspect, the invention provides a kit containing a dose of a SOS1 inhibitor, or a pharmaceutically acceptable salt or a pharmaceutical composition thereof and an MEK inhibitor compound or a pharmaceutically acceptable salt or a pharmaceutical composition thereof in an amount effective to inhibit proliferation of cancer cells in a subject. The kit in some cases includes an insert with instructions for administration of a SOS1 inhibitor, or a pharmaceutically acceptable salt or a pharmaceutical composition thereof and a MEK inhibitor compound or a pharmaceutically acceptable salt or a pharmaceutical composition thereof. The insert may provide a user with one set of instructions for using the a SOS1 inhibitor or a pharmaceutically acceptable salt or a pharmaceutical composition thereof in combination with the MEK inhibitor compound or a pharmaceutically acceptable salt or a pharmaceutical composition thereof.

In some aspects of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### BRIEF DESCRIPTION OF THE DRAWINGS

FIG. 1 depicts average tumor volumes (mm3) of LN229 tumor bearing mice treated with single agents (MRTX-0902, VS-6766) and in combination

FIG. 2 depicts average tumor volumes (mm3) of NCI-H1435 tumor bearing mice treated with single agents (MRTX-0902, VS-6766) and in combination

### DETAILED DESCRIPTION OF THE INVENTION

The present invention relates to combination therapies for treating SOS1-associated cancers. In particular, the present invention relates to methods of treating cancer in a subject in need thereof, comprising administering to the subject a therapeutically effective amount of a combination of a SOS1 inhibitor or a pharmaceutically acceptable salt or a pharmaceutical composition thereof, and a MEK inhibitor or a pharmaceutically acceptable salt or a pharmaceutical composition thereof, pharmaceutical compositions comprising therapeutically effective amounts of the inhibitors, kits comprising the compositions and methods of use therefor.

Combinations of the SOS1 inhibitor, or a pharmaceutically acceptable salt or a pharmaceutical composition thereof, with a MEK inhibitor, or a pharmaceutically acceptable salt or a pharmaceutical composition thereof, synergistically increase the potency of the SOS1 inhibitor against cancer cells that express SOS1 thereby increasing the efficacy and therapeutic index of the SOS1 inhibitor or pharmaceutically acceptable salts thereof.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “MEK” refers to mitogen-activated protein kinase kinase. A MEK inhibitor is a chemical or drug that inhibits the mitogen-activated protein kinase kinase enzymes MEK1 and/or MEK. MEK inhibitors can be used to affect the MAPK/ERK pathway which is often overactive in some cancers.

As used herein, an “MEK” refers to compounds such as VS-6766, binimetinib, cobimetinib, selumetinib, trametinib, PD-325901, CI-1040 or TAK733 and other small and large molecule MEK, or pharmaceutically acceptable salts thereof. These compounds are capable of negatively modulating or inhibiting all or a portion of the enzymatic activity of MEK, or a mutated form of MEK.

A “MEK-associated disease or disorder” as used herein refers to diseases or disorders associated with or mediated by or having a EGFR mutation or over-expression.

As used herein, “SOS1” refers to the Son of sevenless homolog 1 protein encoded by the SOS1 gene that is involved in signaling through RAS pathways.

As used herein, a “SOS1 inhibitor” refers to a compound that is capable of negatively modulating or inhibiting all or a portion of the interaction between KRAS and SOS1.

A “SOS1-associated disease or disorder” as used herein refers to diseases or disorders associated with or mediated by SOS1. A non-limiting example of a SOS1-associated disease or disorder is a SOS1-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12C mutation (e.g., as determined using a regulatory agency-approved, e.g., FDA-approved, assay or kit). In some embodiments, the subject has a tumor that is positive for a KRas G12C mutation (e.g., as determined using a regulatory agency-approved assay or kit). The subject can be a subject with a tumor(s) that is positive for a KRas G12C mutation (e.g., identified as positive using a regulatory agency-approved, e.g., FDA-approved, assay or kit). The subject can be a subject whose tumors have a KRas G12C mutation (e.g., where the tumor is identified as such using a regulatory agency-approved, e.g., FDA-approved, kit or assay). In some embodiments, the subject is suspected of having a KRas G12C gene-associated cancer. In some embodiments, the subject has a clinical record indicating that the subject has a tumor that has a KRas G12C mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments of any of the methods or uses described herein, an assay is used to determine whether the patient has SOS1 over-expression using a sample (e.g., a biological sample or a biopsy sample such as a paraffin-embedded biopsy sample) from a patient (e.g., a patient suspected of having a SOS1-associated cancer, a patient having one or more symptoms of a SOS1-associated cancer, and/or a patient that has an increased risk of developing a SOS1-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR, quantitative real-time RT-PCR, allele-specific genotyping or ddPCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

As used herein, “an effective amount” of a compound is an amount that is sufficient to negatively modulate or inhibit the activity of the desired target, i.e., SOS1 or MEK. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of SOS1 or MEK. Such amount may be administered as a single dosage or may be administered according to a regimen, whereby it is effective.

As used herein, a “therapeutically effective amount of a combination” of two compounds is an amount that together synergistically increases the activity of the combination in comparison to the therapeutically effective amount of each compound in the combination, i.e., more than merely additive. Alternatively, in vivo, the therapeutically effective amount of the combination of a SOS1 inhibitor, or a pharmaceutically acceptable salt or a pharmaceutical composition thereof, and the MEK inhibitor compound or a pharmaceutically acceptable salt or a pharmaceutical composition thereof, results in an increased duration of overall survival (“OS”) in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the MEK inhibitor or a pharmaceutically acceptable salt or a pharmaceutical composition thereof, results in an increased duration of progression-free survival (“PFS”) in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the MEK inhibitor or a pharmaceutically acceptable salt or a pharmaceutical composition thereof, results in increased tumor regression in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the MEK inhibitor or a pharmaceutically acceptable salt or a pharmaceutical composition thereof, results in increased tumor growth inhibition in subjects relative to treatment with only the MEK inhibitor. In one embodiment, the therapeutically effective amount of the combination of a SOS1 inhibitor, or a pharmaceutically acceptable salt or a pharmaceutical composition thereof, and the MEK inhibitor or a pharmaceutically acceptable salt or a pharmaceutical composition thereof, results in an improvement in the duration of stable disease in subjects compared to treatment with only the SOS1 inhibitor, or only the MEK inhibitor. The amount of each compound in the combination may be the same or different than the therapeutically effective amount of each compound when administered alone as a monotherapy as long as the combination is synergistic. Such amounts may be administered as a single dosage or may be administered according to a regimen, whereby it is effective.

As used herein, “treatment” means any manner in which the symptoms or pathology of a condition, disorder or disease are ameliorated or otherwise beneficially altered. Treatment also encompasses any pharmaceutical use of the compositions herein.

As used herein, “amelioration” of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of a MEK inhibitor or a SOS1 inhibitor or a pharmaceutically acceptable salt thereof, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

As used herein, the term “contacting” refers to the bringing together of indicated moieties in an in vitro system or an in vivo system. For example, “contacting” a cancer cell includes the administration of a combination provided herein to an individual or subject, such as a human, having KRas G12C, as well as, for example, introducing a combination provided herein into a sample containing a cellular or purified preparation containing KRas G12C.

### MEK Inhibitor Compounds

In one aspect of the invention, provided herein are methods of treating cancer in a subject in need thereof, comprising administering to the subject a therapeutically effective amount of a combination of a SOS1 inhibitor, or a pharmaceutically acceptable salt or a pharmaceutical composition thereof, and a MEK inhibitor or a pharmaceutically acceptable salt or a pharmaceutical composition thereof.

In one embodiment, the MEK inhibitor is:

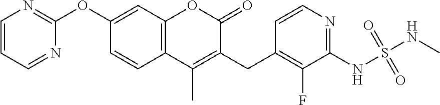 [SMILES: CNS(=O)(=O)Nc1nccc(Cc2c(C)c3ccc(Oc4ncccn4)cc3oc2=O)c1F]

(also referred to as VS-6766), or a pharmaceutically acceptable salt thereof.

In one embodiment, the MEK inhibitor is:

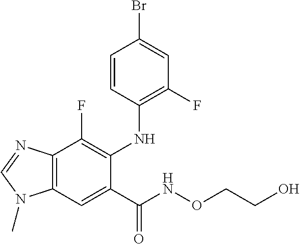 [SMILES: Cn1cnc2c(F)c(Nc3ccc(Br)cc3F)c(C(=O)NOCCO)cc21]

(also referred to as binimetinib), or a pharmaceutically acceptable salt thereof.

In one embodiment, the MEK inhibitor is.

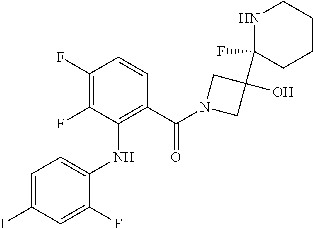 [SMILES: O=C(c1ccc(F)c(F)c1Nc1ccc(I)cc1F)N1CC(O)([C@]2(F)CCCCN2)C1]

(also referred to as cobimetinib), or a pharmaceutically acceptable salt thereof.

In one embodiment, the MEK inhibitor is:

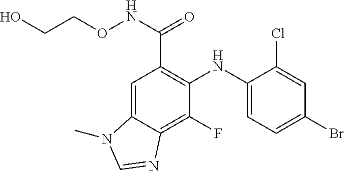 [SMILES: Cn1cnc2c(F)c(Nc3ccc(Br)cc3Cl)c(C(=O)NOCCO)cc21]

(also referred to as selumetinib), or a pharmaceutically acceptable salt thereof.

In one embodiment, the MEK inhibitor is.

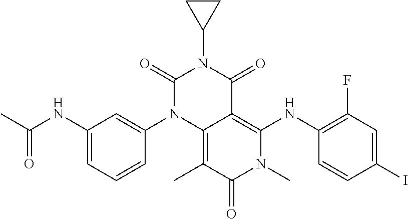 [SMILES: CC(=O)Nc1cccc(-n2c(=O)n(C3CC3)c(=O)c3c(Nc4ccc(I)cc4F)n(C)c(=O)c(C)c32)c1]

(also referred to as trametinib), or a pharmaceutically acceptable salt thereof.

In one embodiment, the MEK inhibitor is (also referred to as PD-325901), or a pharmaceutically acceptable salt thereof.

In one embodiment, the MEK inhibitor is CI-1040, or a pharmaceutically acceptable salt thereof.

In one embodiment, the MEK inhibitor is TAK733, or a pharmaceutically acceptable salt thereof.

The MEK inhibitors used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In one embodiment, the KRas G12C inhibitor compound adagrasib used in the methods include salts of the above compounds, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Methods for manufacturing the KRas G12C inhibitors disclosed herein are generally well known.

### SOS1 Inhibitor Compounds

In one embodiment, the SOS1 inhibitor is a compound selected from (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino)pyrrolidin-1-yl)-4-methylpyrido[3,4-d]pyridazin-1-yl)amino)ethyl)-2-methylbenzonitrile, (R)-3-(1-((6-fluoro-4-methyl-7-(4-methylpiperazin-1-yl)phthalazin-1-yl)amino)ethyl)-2-methylbenzonitrile, or a pharmaceutically acceptable salt thereof; or selected from the compounds described in ______ as described in greater detail herein.

In another embodiment the SOS1 inhibitor is a compound of the formula:

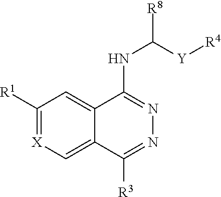

or a pharmaceutically acceptable salt thereof, wherein: R¹ is hydrogen, hydroxyl, C1-C6 alkyl, alkoxy, —N(R⁶)₂, —NR⁶C(O)R⁶, —C(O)N(R⁶)₂, —SO₂alkyl, —SO₂NR⁶alkyl, cycloalkyl, -Q-heterocyclyl, aryl, or heteroaryl, wherein the cycloalkyl, the heterocyclyl, the aryl, and the heteroaryl are each optionally substituted with one or more R²; each Q is independently a bond, O, or NR⁶; X is N or CR⁷; each R² is independently hydroxy, halogen, cyano, hydroxyalkyl, haloalkyl, alkoxy, —N(R⁶)₂, —SO₂alkyl, —NR⁶C(O)C1-C3 alkyl, —C(O)cycloalkyl, —C(O)heretocyclyl or aryl, wherein the cycloalkyl, the heterocyclyl or the aryl are each optionally substituted with one or more R¹¹; R³ is hydrogen, C1-C6 alkyl, alkoxy, —N(R¹⁰)₂, cycloalkyl, haloalkyl, heterocyclyl, aryl, or heteroaryl, wherein the C1-C6 alkyl, the cycloalkyl, the heterocyclyl, the aryl, and the heteroaryl are each optionally substituted with one or more R⁹; Y is a bond or heteroarylene; R⁴ is aryl or heteroaryl, each optionally substituted with one or more R⁵; each R⁵ is independently hydroxy, halogen, cyano, hydroxyalkyl, alkoxy, C1-C3 alkyl, haloalkyl, —N(R⁶)₂, -L-N(R⁶)₂ or -SO₂alkyl; L is C1-C3 alkylene; each R⁶ is independently hydrogen, C1-C3 alkyl, haloalkyl, or cycloalkyl; R⁷ is hydrogen, cyano, or alkoxy; R⁸ is C1-C2 alkyl or halo-C1-C2 alkyl; each R⁹ is independently hydroxy, halogen, amino, cyano, alkoxy, or C1-C3 alkyl; each R¹⁰ is independently hydrogen, C1-C3 alkyl or cycloalkyl; and each R¹¹ is independently C1-C3 alkyl or haloalkyl. These compounds include, but are not limited to, all of the exemplary compounds recited in WO2021/127429, WO2021/173524, WO2022/026465, U.S. provisional patent application 63/213,112 (and corresponding national and international applications and publications) and as described in greater detail herein, including in particular: (R)-2-methyl-3-(1-((4-methyl-7-morpholinopyrido[3,4-d]pyridazin-1-yl)amino)ethyl)benzonitrile.

In another embodiment, the SOS1 inhibitor is a compound selected from:

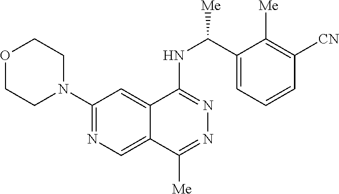 [SMILES: Cc1c(C#N)cccc1[C@@H](C)Nc1nnc(C)c2cnc(N3CCOCC3)cc12]

(R)-2-methyl-3-(1-((4-methyl-7-morpholinopyrido[3,4-d]pyridazin-1-yl)amino)ethyl)benzonitrile,

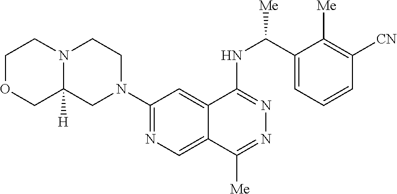 [SMILES: Cc1c(C#N)cccc1[C@@H](C)Nc1nnc(C)c2cnc(N3CCN4CCOC[C@@H]4C3)cc12]

3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile,

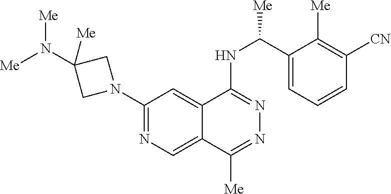 [SMILES: Cc1c(C#N)cccc1[C@@H](C)Nc1nnc(C)c2cnc(N3CC(C)(N(C)C)C3)cc12]

(R)-3-(1-((7-(3-(dimethylamino)-3-methylazetidin-1-yl)-4-methylpyrido[3,4-d]pyridazin-1-yl)amino)ethyl)-2-methylbenzonitrile, and

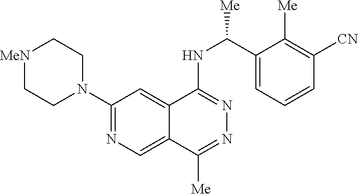 [SMILES: Cc1c(C#N)cccc1[C@@H](C)Nc1nnc(C)c2cnc(N3CCN(C)CC3)cc12]

(R)-2-methyl-3-(1-((4-methyl-7-(4-methylpiperazin-1-yl)pyrido[3,4-d]pyridazin-1-yl)amino)ethyl)benzonitrile,

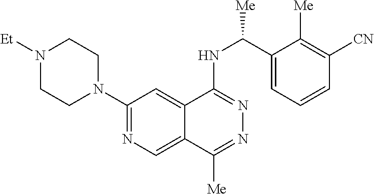 [SMILES: CCN1CCN(c2cc3c(N[C@H](C)c4cccc(C#N)c4C)nnc(C)c3cn2)CC1]

(R)-3-(1-((7-(4-ethylpiperazin-1-yl)-4-methylpyrido[3,4-d]pyridazin-1-yl)amino)ethyl)-2-methylbenzonitrile,

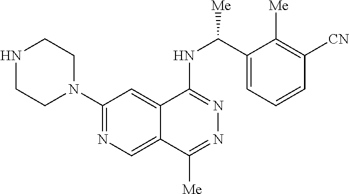 [SMILES: Cc1c(C#N)cccc1[C@@H](C)Nc1nnc(C)c2cnc(N3CCNCC3)cc12]

(R)-2-methyl-3-(1-((4-methyl-7-(piperazin-1-yl)pyrido[3,4-d]pyridazin-1-yl)amino)ethyl)benzonitrile,

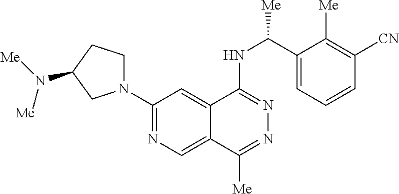 [SMILES: Cc1c(C#N)cccc1[C@@H](C)Nc1nnc(C)c2cnc(N3CC[C@H](N(C)C)C3)cc12]

3-((R)-1-((7-((S)-3-(dimethylamino)pyrrolidin-1-yl)-4-methylpyrido[3,4-d]pyridazin-1-yl)amino)ethyl)-2-methylbenzonitrile, and

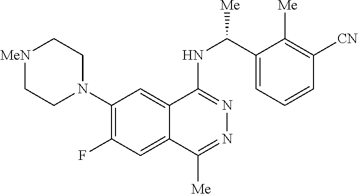 [SMILES: Cc1c(C#N)cccc1[C@@H](C)Nc1nnc(C)c2cc(F)c(N3CCN(C)CC3)cc12]

(R)-3-(1-((6-fluoro-4-methyl-7-(4-methylpiperazin-1-yl)phthalazin-1-yl)amino)ethyl)-2-methylbenzonitrile, and pharmaceutically acceptable salts thereof.

The SOS1 inhibitors used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In one embodiment, the SOS1 inhibitor compound includes its salts, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Methods for manufacturing the SOS1 inhibitors disclosed herein are known. For example, commonly owned applications WO2021/127429, WO2021/173524, WO2022/026465, U.S. provisional patent application 63/213,112 (and corresponding national and international applications and publications) describe general reaction schemes for preparing compounds including adagrasib and also provide detailed synthetic routes for the preparation of these compounds.

### Pharmaceutical Compositions

The SOS1 inhibitors and the MEK inhibitor or pharmaceutically acceptable salts thereof may be formulated into pharmaceutical compositions.

In another aspect, the invention provides pharmaceutical compositions comprising a SOS1 inhibitor, or a pharmaceutically acceptable salt thereof, and a MEK inhibitor, or a pharmaceutically acceptable salt thereof and a pharmaceutically acceptable carrier, excipient, or diluent that may be used in the methods disclosed herein. The SOS1 inhibitor, or a pharmaceutically acceptable salt thereof, and MEK inhibitor, or a pharmaceutically acceptable salt thereof may be independently formulated by any method well known in the art and may be prepared for administration by any route, including, without limitation, parenteral, oral, sublingual, transdermal, topical, intranasal, intratracheal, or intrarectal. In certain embodiments, the SOS1 inhibitor, or a pharmaceutically acceptable salt thereof, and/or the KRas G12C inhibitor, or a pharmaceutically acceptable salt thereof, is/are administered intravenously in a hospital setting.

In one embodiment, administration of one or both therapeutic components may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

As used herein, the term “pharmaceutically acceptable salt” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising a SOS1 inhibitor, or a pharmaceutically acceptable salt or a pharmaceutical composition thereof and a MEK inhibitor, or a pharmaceutically acceptable salt or a pharmaceutical composition thereof, may be used in the methods of use described herein.

### Co-Administration

The SOS1 inhibitor, or a pharmaceutically acceptable salt thereof, and the MEK inhibitor, or a pharmaceutically acceptable salt thereof, can be formulated into separate or individual dosage forms which can be co-administered one after the other. Another option is that if the route of administration is the same (e.g. oral) two active compounds can be formulated into a single form for co-administration, both methods of co-administration, however, being part of the same therapeutic treatment or regimen.

The pharmaceutical compositions comprising a SOS1 inhibitor, or a pharmaceutically acceptable salt thereof, and/or a MEK inhibitor, or a pharmaceutically acceptable salt thereof, for use in the methods may be for simultaneous, separate or sequential use. In one embodiment, the SOS1 inhibitor or a pharmaceutically acceptable salt thereof, is administered prior to administration of the MEK inhibitor or a pharmaceutically acceptable salt thereof. In another embodiment, the SOS1 inhibitor, or a pharmaceutically acceptable salt thereof, is administered after administration of the MEK inhibitor or a pharmaceutically acceptable salt thereof. In another embodiment, the SOS1 inhibitor, or a pharmaceutically acceptable salt thereof, is administered at about the same time as administration of the MEK inhibitor compound or a pharmaceutically acceptable salt thereof. Separate administration of each inhibitor, at different times and by different routes, in some cases would be advantageous. Thus, the components in the combination i.e. the MEK inhibitor or a pharmaceutically acceptable salt thereof and the SOS1 inhibitor, or a pharmaceutically acceptable salt thereof, need not be necessarily administered at essentially the same time or in any order.

Oncology drugs are typically administered at the maximum tolerated dose (“MTD”), which is the highest dose of drug that does not cause unacceptable side effects. In one embodiment, the MEK inhibitor, or a pharmaceutically acceptable salt or a pharmaceutical composition thereof and the SOS1 inhibitor, or a pharmaceutically acceptable salt or a pharmaceutical composition thereof, are each dosed at their respective MTDs. In one embodiment, the MEK inhibitor, or a pharmaceutically acceptable salt or a pharmaceutical composition thereof, is dosed at its MTD and the SOS1 inhibitor, or a pharmaceutically acceptable salt or a pharmaceutical composition thereof, is dosed in an amount less than its MTD. In one embodiment, the MEK inhibitor, or a pharmaceutically acceptable salt or a pharmaceutical composition thereof, is dosed at an amount less than its MTD and the SOS1 inhibitor, or a pharmaceutically acceptable salt or a pharmaceutical composition thereof, is dosed at its MTD. In one embodiment, the MEK inhibitor, or a pharmaceutically acceptable salt or a pharmaceutical composition thereof and the SOS1 inhibitor, or a pharmaceutically acceptable salt or a pharmaceutical composition thereof are each dosed at less than their respective MTDs. The administration can be so timed that the peak pharmacokinetic effect of one compound coincides with the peak pharmacokinetic effect of the other.

In one embodiment, a single dose of MEK inhibitor or a pharmaceutically acceptable salt or a pharmaceutical composition thereof, is administered per day (i.e., in about 24 hour intervals) (i.e., QD). In another embodiment, two doses of the MEK inhibitor or a pharmaceutically acceptable salt or a pharmaceutical composition thereof, are administered per day (i.e., BID). In another embodiment, three doses of the MEK inhibitor or a pharmaceutically acceptable salt or a pharmaceutical composition thereof, are administered per day (i.e., TID).

In one embodiment, the SOS1 inhibitor, or a pharmaceutically acceptable salt or a pharmaceutical composition thereof, is administered QD. In another embodiment the SOS1 inhibitor, or a pharmaceutically acceptable salt or a pharmaceutical composition thereof, are administered BID. In another embodiment, the SOS1 inhibitor, or a pharmaceutically acceptable salt or a pharmaceutical composition thereof, of the invention are administered TID.

In one embodiment, a single dose of MEK inhibitor or a pharmaceutically acceptable salt or a pharmaceutical composition thereof, and SOS1 inhibitor, or a pharmaceutically acceptable salt or a pharmaceutical composition thereof are each administered once daily.

Examples of SOS1 inhibitors suitable for the provided compositions and methods include those mentioned herein, for example (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino)pyrrolidin-1-yl)-4-methylpyrido[3,4-d]pyridazin-1-yl)amino)ethyl)-2-methylbenzonitrile, and (R)-3-(1-((6-fluoro-4-methyl-7-(4-methylpiperazin-1-yl)phthalazin-1-yl)amino)ethyl)-2-methylbenzonitrile.

### Combination Therapies

In one aspect of the invention, provided herein are methods of treating cancer in a subject in need thereof, comprising administering to the subject a therapeutically effective amount of a combination of a SOS1 inhibitor, or a pharmaceutically acceptable salt or a pharmaceutical composition thereof, and the MEK inhibitor or a pharmaceutically acceptable salt or a pharmaceutical composition thereof. In one embodiment, the cancer is a SOS1-associated cancer. In one embodiment, the SOS1-associated cancer is lung cancer.

In yet another aspect, the invention provides for methods for increasing the sensitivity of a cancer cell to a SOS1 inhibitor, comprising contacting the cancer cell with an effective amount of a combination of the SOS1 inhibitor, such as MRTX0902, or a pharmaceutically acceptable salt or a pharmaceutical composition thereof, and a MEK inhibitor, such as VS-6766, or a pharmaceutically acceptable salt or a pharmaceutical composition thereof, wherein the MEK inhibitor synergistically increases the sensitivity of the cancer cell to the SOS1 inhibitor. In one embodiment, the contacting is in vitro. In one embodiment, the contacting is in vivo.

In one embodiment, the combination therapy comprises a combination of a compound having the formula:

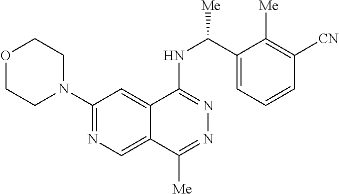 [SMILES: Cc1c(C#N)cccc1[C@@H](C)Nc1nnc(C)c2cnc(N3CCOCC3)cc12]

(MRTX0902) or a pharmaceutically acceptable salt thereof, and an MEK inhibitor.

In one such embodiment, the MEK inhibitor is VS-6766.

In one such embodiment, the MEK inhibitor is binimetinib.

In one such embodiment, the MEK inhibitor is cobimetinib.

In one such embodiment, the MEK inhibitor is selumetinib.

In one such embodiment, the MEK inhibitor is trametinib.

In one such embodiment, the MEK inhibitor is PD-325901.

In one such embodiment, the MEK inhibitor is CI-1040 or TAK733.

In one embodiment, the combination therapy comprises a combination VS-6766 and a SOS1 inhibitor.

In one such embodiment, the SOS1 inhibitor is:

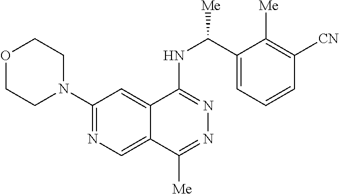 [SMILES: Cc1c(C#N)cccc1[C@@H](C)Nc1nnc(C)c2cnc(N3CCOCC3)cc12]

(R)-2-methyl-3-(1-((4-methyl-7-morpholinopyrido[3,4-d]pyridazin-1-yl)amino)ethyl)benzonitrile (MRTX0902).

In another such embodiment, the SOS1 inhibitor is:

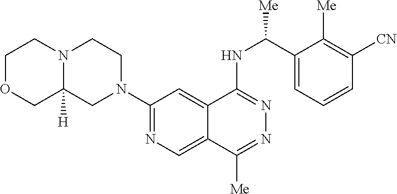 [SMILES: Cc1c(C#N)cccc1[C@@H](C)Nc1nnc(C)c2cnc(N3CCN4CCOC[C@@H]4C3)cc12]

3-((R)-1-((7-((s)-hexahydropyrazino[2,1-c][1,4]oxazin-8(1H)-yl)-4-methylpyrido[3,4-d]pyridazin-1-yl)amino)ethyl)-2-methylbenzonitrile

In yet another such embodiment, the SOS1 inhibitor is:

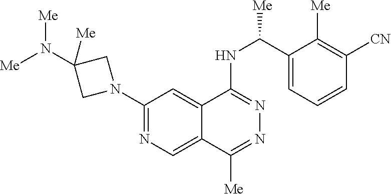 [SMILES: Cc1c(C#N)cccc1[C@@H](C)Nc1nnc(C)c2cnc(N3CC(C)(N(C)C)C3)cc12]

(R)-3-(1-((7-(3-(dimethylamino)-3-methylazetidin-yl)-4-methylpyrido[3,4-d]pyridazin-1-yl)amino)ethyl)-2-methylbenzonitrile.

In still another such embodiment, the SOS1 inhibitor is:

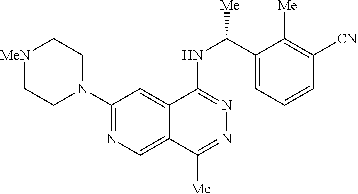 [SMILES: Cc1c(C#N)cccc1[C@@H](C)Nc1nnc(C)c2cnc(N3CCN(C)CC3)cc12]

(R)-2-methyl-3-(1-((4-methyl-7-(4-methylpiperazin-1-yl)pyrido[3,4-d]pyridazin-1-yl)amino)ethyl)benzonitrile.

In another embodiment, the SOS1 inhibitor is:

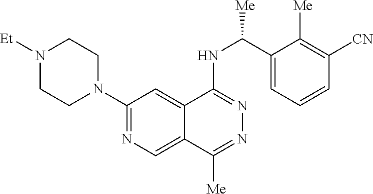 [SMILES: CCN1CCN(c2cc3c(N[C@H](C)c4cccc(C#N)c4C)nnc(C)c3cn2)CC1]

(R)-3-(1-((7-(4-ethylpiperazin-1-yl)-4-methylpyrido[3,4-d]pyridazin-1-yl)amino)ethyl)-2-methylbenzonitrile.

In another embodiment, the SOS1 inhibitor is:

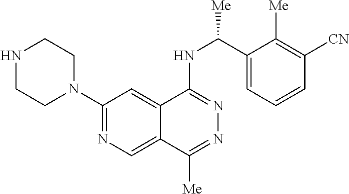 [SMILES: Cc1c(C#N)cccc1[C@@H](C)Nc1nnc(C)c2cnc(N3CCNCC3)cc12]

(R)-2-methyl-3-(1-((4-methyl-7-(piperazin-1-yl)pyrido[3,4-d]pyridazin-1-yl)amino)ethyl)benzonitrile.

In another embodiment, the SOS1 inhibitor is:

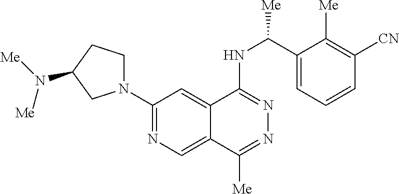 [SMILES: Cc1c(C#N)cccc1[C@@H](C)Nc1nnc(C)c2cnc(N3CC[C@H](N(C)C)C3)cc12]

3-((R)-1-((7-((S)-3-(dimethylamino)pyrrolidin-1-yl)-4-methylpyrido[3,4-d]pyridazin-1-yl)amino)ethyl)-2-methylbenzonitrile.

In another embodiment, the SOS1 inhibitor is:

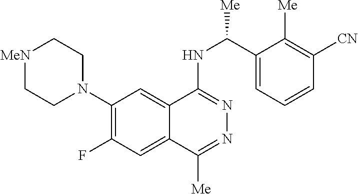 [SMILES: Cc1c(C#N)cccc1[C@@H](C)Nc1nnc(C)c2cc(F)c(N3CCN(C)CC3)cc12]

(R)-3-(1-((6-fluoro-4-methyl-7-(4-methylpiperazin-1-yl)phthalazin-1-yl)amino)ethyl)-2-methylbenzonitrile.

The methods described herein are designed to inhibit undesired cellular proliferation resulting from enhanced EGFR activity within the cell. The degree of inhibitory activity of the SOS1 inhibitor—EGFR inhibitor combination in cells may be monitored, for example, by measuring cell viability and functional inhibition of both RAF/MEK/ERK and PI3K/AKT effector pathway signaling (amounts of phosphorylated ERK and AKT, respectively) to assess the effectiveness of treatment and dosages may be adjusted accordingly by the attending medical practitioner.

The compositions and methods provided herein may be used for the treatment of a SOS1-associated cancer in a subject in need thereof, comprising administering to said subject a therapeutically effective amount of a combination of a SOS1 inhibitor, or a pharmaceutically acceptable salt or a pharmaceutical composition thereof, and a MEK inhibitor or a pharmaceutically acceptable salt or a pharmaceutical composition thereof, wherein the MEK inhibitor synergistically increases the sensitivity of the SOS1-associated cancer to the SOS1 inhibitor. In one embodiment, the SOS1 associated cancer is a cancer with genetic alterations of the MAPK pathway. In one embodiment, the SOS1 associated cancer is a cancer mediated by SOS1. In one embodiment, the SOS1-associated cancer is lung cancer. In one embodiment, the SOS1-associated cancer is brain cancer.

In one embodiment, the therapeutically effective amount of the combination of a SOS1 inhibitor, or a pharmaceutically acceptable salt or a pharmaceutical composition thereof, and a MEK inhibitor or a pharmaceutically acceptable salt or a pharmaceutical composition thereof, results in an increased duration of overall survival (“OS”) in subjects relative to treatment with only the SOS1 inhibitor. In one embodiment, the therapeutically effective amount of the combination of a SOS1 inhibitor, or a pharmaceutically acceptable salt or a pharmaceutical composition thereof, and a MEK inhibitor or a pharmaceutically acceptable salt or a pharmaceutical composition thereof, results in an increased duration of progression-free survival (“PF S”) in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the MEK inhibitor or a pharmaceutically acceptable salt or a pharmaceutical composition thereof, results in increased tumor regression in subjects relative to treatment with only the SOS1 inhibitor. In one embodiment, the therapeutically effective amount of the combination of a SOS1 inhibitor, or a pharmaceutically acceptable salt or a pharmaceutical composition thereof, and MEK inhibitor or a pharmaceutically acceptable salt or a pharmaceutical composition thereof, results in increased tumor growth inhibition in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the MEK inhibitor or a pharmaceutically acceptable salt or a pharmaceutical composition thereof, results in an improvement in the duration of stable disease in subjects compared to treatment with only the SOS1 inhibitor.

In one embodiment, the SOS1 inhibitor is selected from (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino)pyrrolidin-1-yl)-4-methylpyrido[3,4-d]pyridazin-1-yl)amino)ethyl)-2-methylbenzonitrile, (R)-3-(1-((6-fluoro-4-methyl-7-(4-methylpiperazin-1-yl)phthalazin-1-yl)amino)ethyl)-2-methylbenzonitrile, or a pharmaceutically acceptable salt thereof.

In another embodiment, the SOS1 inhibitor, or a pharmaceutically acceptable salt or a pharmaceutical composition thereof, is administered in combination with the MEK inhibitor, or a pharmaceutically acceptable salt or a pharmaceutical composition thereof, once disease progression has been observed for SOS1 monotherapy, in which the combination therapy results in enhanced clinical benefit for the patient by increasing OS, PFS, tumor regression, tumor growth inhibition or the duration of stable disease in the patient.

In one embodiment, the therapeutic combination comprises therapeutically effective amounts of VS-6766 and (R)-2-methyl-3-(1-((4-methyl-7-morpholinopyrido[3,4-d]pyridazin-1-yl)amino)ethyl)benzonitrile, or a pharmaceutically acceptable salt thereof.

In another embodiment, the therapeutic combination comprises therapeutically effective amounts of VS-6766 and 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile, or a pharmaceutically acceptable salt thereof.

In another embodiment, the therapeutic combination comprises therapeutically effective amounts of VS-6766 and (R)-3-(1-((7-(3-(dimethylamino)-3-methylazetidin-1-yl)-4-methylpyrido[3,4-d]pyridazin-1-yl)amino)ethyl)-2-methylbenzonitrile, or a pharmaceutically acceptable salt thereof.

In another embodiment, the therapeutic combination comprises therapeutically effective amounts of VS-6766 and (R)-2-methyl-3-(1-((4-methyl-7-(4-methylpiperazin-1-yl)pyrido[3,4-d]pyridazin-1-yl)amino)ethyl)benzonitrile, or a pharmaceutically acceptable salt thereof.

In another embodiment, the therapeutic combination comprises therapeutically effective amounts of VS-6766 and (R)-3-(1-((7-(4-ethylpiperazin-1-yl)-4-methylpyrido[3,4-d]pyridazin-1-yl)amino)ethyl)-2-methylbenzonitrile, or a pharmaceutically acceptable salt thereof.

In another embodiment, the therapeutic combination comprises therapeutically effective amounts of VS-6766 and (R)-2-methyl-3-(1-((4-methyl-7-(piperazin-1-yl)pyrido[3,4-d]pyridazin-1-yl)amino)ethyl)benzonitrile, or a pharmaceutically acceptable salt thereof.

In another embodiment, the therapeutic combination comprises therapeutically effective amounts of VS-6766 and 3-((R)-1-((7-((S)-3-(dimethylamino)pyrrolidin-1-yl)-4-methylpyrido[3,4-d]pyridazin-1-yl)amino)ethyl)-2-methylbenzonitrile, or a pharmaceutically acceptable salt thereof.

In another embodiment, the therapeutic combination comprises therapeutically effective amounts of VS-6766 and (R)-3-(1-((6-fluoro-4-methyl-7-(4-methylpiperazin-1-yl)phthalazin-1-yl)amino)ethyl)-2-methylbenzonitrile, or a pharmaceutically acceptable salt thereof.

The compositions and methods provided herein may be used for the treatment of a wide variety of cancers including tumors such as lung, colorectal, pancreas, prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

Also provided herein is a method for treating cancer in a subject in need thereof, the method comprising (a) determining that cancer is associated with SOS1 over-expression (e.g., as determined using a regulatory agency-approved, e.g., FDA-approved, assay or kit); and (b) administering to the patient a therapeutically effective amount of a combination of a SOS1 inhibitor, or a pharmaceutically acceptable salt or a pharmaceutical composition thereof, and a MEK inhibitor or a pharmaceutically acceptable salt or a pharmaceutical composition thereof, wherein the MEK inhibitor synergistically increases the sensitivity of the SOS1-associated cancer to the SOS1 inhibitor. In one embodiment, the MEK inhibitor is selected from VS-6766, binimetinib, cobimetinib, selumetinib, trametinib, PD-325901, CI-1040 and CI-1040 or TAK733 and other small and large molecule MEK inhibitors, and the SOS1 inhibitor is selected from: (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino)pyrrolidin-1-yl)-4-methylpyrido[3,4-d]pyridazin-1-yl)amino)ethyl)-2-methylbenzonitrile, (R)-3-(1-((6-fluoro-4-methyl-7-(4-methylpiperazin-1-yl)phthalazin-1-yl)amino)ethyl)-2-methylbenzonitrile, or a pharmaceutically acceptable salt thereof.

In one embodiment, the therapeutic combination comprises therapeutically effective amounts of (R)-2-methyl-3-(1-((4-methyl-7-morpholinopyrido[3,4-d]pyridazin-1-yl)amino)ethyl)benzonitrile or a pharmaceutically acceptable salt thereof.

In a further embodiment, the therapeutic combination comprises therapeutically effective amounts of 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile or a pharmaceutically acceptable salt thereof.

In a further embodiment, the therapeutic combination comprises therapeutically effective amounts of (R)-3-(1-((7-(3-(dimethylamino)-3-methylazetidin-1-yl)-4-methylpyrido[3,4-d]pyridazin-1-yl)amino)ethyl)-2-methylbenzonitrile or a pharmaceutically acceptable salt thereof.

In a further embodiment, the therapeutic combination comprises therapeutically effective amounts of (R)-2-methyl-3-(1-((4-methyl-7-(4-methylpiperazin-1-yl)pyrido[3,4-d]pyridazin-1-yl)amino)ethyl)benzonitrile or a pharmaceutically acceptable salt thereof.

In a further embodiment, the therapeutic combination comprises therapeutically effective amounts of (R)-3-(1-((7-(4-ethylpiperazin-1-yl)-4-methylpyrido[3,4-d]pyridazin-1-yl)amino)ethyl)-2-methylbenzonitrile or a pharmaceutically acceptable salt thereof.

In a further embodiment, the therapeutic combination comprises therapeutically effective amounts of (R)-2-methyl-3-(1-((4-methyl-7-(piperazin-1-yl)pyrido[3,4-d]pyridazin-1-yl)amino)ethyl)benzonitrile or a pharmaceutically acceptable salt thereof.

In a further embodiment, the therapeutic combination comprises therapeutically effective amounts of 3-((R)-1-((7-((S)-3-(dimethylamino)pyrrolidin-1-yl)-4-methylpyrido[3,4-d]pyridazin-1-yl)amino)ethyl)-2-methylbenzonitrile or a pharmaceutically acceptable salt thereof.

In a further embodiment, the therapeutic combination comprises therapeutically effective amounts of (R)-3-(1-((6-fluoro-4-methyl-7-(4-methylpiperazin-1-yl)phthalazin-1-yl)amino)ethyl)-2-methylbenzonitrile or a pharmaceutically acceptable salt thereof.

In one embodiment, the SOS1 inhibitor, the MEK inhibitor, or both, is/are administered as a tablet or capsule during the period of time. In one embodiment, the tablet or capsule formulation of the SOS1 inhibitor and/or the EGR inhibitor comprises one or more of: about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg and about 2000 mg. In one embodiment, the SOS1 inhibitor and/or the MEK inhibitor is orally administered once a day (QD) on a daily basis during a period of time. In one embodiment SOS1 inhibitor and/or the MEK inhibitor is orally administered twice a day (BID) on a daily basis during a period of time.

In one embodiment, the SOS1 inhibitor and/or the MEK inhibitor is/are orally administered in the amount of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 25, about 50, about 75, about 100, about 150, about 200, about 250, about 300, about 350, about 400, about 450, or about 500 mg) over a period of time. In one embodiment, the SOS1 inhibitor and/or the MEK inhibitor is/are orally administered twice a day (BID) on a daily basis during a period of time. In one embodiment adagrasib is orally administered twice a day (BID) on a daily basis during a period of time.

In one embodiment, the combination therapy comprises oral administration of the SOS1 inhibitor and/or the MEK inhibit, each independently once or twice a day on a daily basis (during a period of time), e.g., in an amount of about 10 mg to about 400 mg (e.g., about 10 mg to about 380 mg, about 10 mg to about 360 mg, about 10 mg to about 340 mg, about 10 mg to about 320 mg, about 10 mg to about 300 mg, about 10 mg to about 280 mg, about 10 mg to about 260 mg, about 10 mg to about 240 mg, about 10 mg to about 220 mg, about 10 mg to about 200 mg, about 10 mg to about 180 mg, about 10 mg to about 160 mg, about 10 mg to about 140 mg, about 10 mg to about 120 mg, about 10 mg to about 100 mg, about 10 mg to about 80 mg, about 10 mg to about 60 mg, about 10 mg to about 40 mg, about 10 mg to about 20 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 400 mg, about 160 mg to about 380 mg, about 160 mg to about 360 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 400 mg, about 300 mg to about 380 mg, about 300 mg to about 360 mg, about 300 mg to about 340 mg, about 300 mg to about 320 mg, about 320 mg to about 400 mg, about 320 mg to about 380 mg, about 320 mg to about 360 mg, about 340 mg to about 360 mg, about 340 mg to about 400 mg, about 340 mg to about 380 mg, about 340 mg to about 360 mg, about 360 mg to about 400 mg, about 360 mg to about 380 mg, about 380 mg to about 400 mg, about 100 mg, about 200 mg, about 300 mg, or about 400 mg). In one embodiment, the KRas G12C inhibitor adagrasib or a pharmaceutically acceptable salt or a pharmaceutical composition thereof is orally administered once daily. In another embodiment, the KRas G12C inhibitor, or a pharmaceutically acceptable salt or a pharmaceutical composition thereof, is orally administered twice daily.

One skilled in the art will recognize that, both in vivo and in vitro trials using suitable, known and generally accepted cell and/or animal models are predictive of the ability of a test compound of the combination or the combination to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### Synergy

In one embodiment, the addition of a MEK inhibitor, or a pharmaceutically acceptable salt or a pharmaceutical composition thereof, synergistically increases the activity of the SOS1 inhibitor compound, for instance MRTX0902 or a pharmaceutically acceptable salt or a pharmaceutical composition thereof, against cancer or cancer cell lines over-expressing SOS1. Any method for determining whether two compounds exhibit synergy may be used for determining the synergistic effect of the combination.

Several mathematical models have been developed to determine whether two compounds act synergistically, i.e., beyond a mere additive effect. For instance, Loewe Additivity (Loewe (1928) Physiol. 27: 47-187), Bliss Independence (Bliss (1939) Ann. Appl. Biol. 26: 585-615), Highest Single Agent, ZIP (Yadav et al (2015) Comput Struct Biotech J 13: 504-513) and other models (Chou & Talalay (1984) Adv Enzyme Regul 22: 27-55. #6382953; and Greco et al. (1995) Pharmacol Rev 47(2): 331-85. #7568331) are well known models in the pharmaceutical industry and may be used to calculate a “synergy score” that indicates whether synergy was detected and the magnitude of such synergy. Combining these synergy scores produces a composite synergy score which may be used to evaluate and characterize a MEK inhibitor such as VS-6766 and a SOS1 inhibitor such as MRTX0902.

In general, the mathematical models use data obtained from single agent values to determine the predicted additive effect of the combination which is compared to the observed effect for the combination. If the observed effect is greater than the predicted effect, the combination is deemed to be synergistic. For example, the Bliss independence model compares the observed combination response (Yo) with the predicted combination response (YP), which was obtained based on the assumption that there is no effect from drug-drug interactions. Typically, the combination effect is declared synergistic if Yo is greater than Y.

In some embodiments, “synergistic effect” as used herein refers to combination of a MEK inhibitor or a pharmaceutically acceptable salt thereof, and a SOS1 inhibitor or a pharmaceutically acceptable salt thereof producing an effect, for example, any of the beneficial or desired results including clinical results or endpoints as described herein, which is greater than the sum of the effect observed when a compound such as one described in the SOS1 patent applications recited herein, for instance MRTX0902, and a MEK inhibitor or a pharmaceutically acceptable salt thereof, for instance VS-6766, are administered alone, acceptable salt thereof.

In one embodiment, the synergistic therapeutic combination comprises therapeutically effective amounts of (R)-2-methyl-3-(1-((4-methyl-7-morpholinopyrido[3,4-d]pyridazin-1-yl)amino)ethyl)benzonitrile and VS-6766. In one embodiment, the synergistic therapeutic combination comprises therapeutically effective amounts of 3-((R)-1-((7-((S)-hexahydropyrazino[2,1-c][1,4]oxazin-8(1H)-yl)-4-methylpyrido[3,4-d]pyridazin-1-yl)amino)ethyl)-2-methylbenzonitrile and VS-6766. In one embodiment, the synergistic therapeutic combination comprises therapeutically effective amounts of (R)-3-(1-((7-(3-(dimethylamino)-3-methylazetidin-1-yl)-4-methylpyrido[3,4-d]pyridazin-1-yl)amino)ethyl)-2-methylbenzonitrile and VS-6766. In one embodiment, the synergistic therapeutic combination comprises therapeutically effective amounts of (R)-2-methyl-3-(1-((4-methyl-7-(4-methylpiperazin-1-yl)pyrido[3,4-d]pyridazin-1-yl)amino)ethyl)benzonitrile and VS-6766. In one embodiment, the synergistic therapeutic combination comprises therapeutically effective amounts of (R)-3-(1-((7-(4-ethylpiperazin-1-yl)-4-methylpyrido[3,4-d]pyridazin-1-yl)amino)ethyl)-2-methylbenzonitrile, and VS-6766. In one embodiment, the synergistic therapeutic combination comprises therapeutically effective amounts of (R)-2-methyl-3-(1-((4-methyl-7-(piperazin-1-yl)pyrido[3,4-d]pyridazin-1-yl)amino)ethyl)benzonitrile, and VS-6766. In one embodiment, the synergistic therapeutic combination comprises therapeutically effective amounts of 3-((R)-1-((7-((S)-3-(dimethylamino)pyrrolidin-1-yl)-4-methylpyrido[3,4-d]pyridazin-1-yl)amino)ethyl)-2-methylbenzonitrile, and VS-6766. In one embodiment, the synergistic therapeutic combination comprises therapeutically effective amounts of (R)-3-(1-((6-fluoro-4-methyl-7-(4-methylpiperazin-1-yl)phthalazin-1-yl)amino)ethyl)-2-methylbenzonitrile and VS-6766.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to ⁴⁰⁰%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### Kits

The present invention also relates to a kit comprising a SOS1 inhibitor, or a pharmaceutically acceptable salt or a pharmaceutical composition thereof (for example, MRTX0902), and a MEK inhibitor or a pharmaceutically acceptable salt or a pharmaceutical composition thereof (for example VS-6766). Also provided is a kit comprising such a SOS1 inhibitor, or a pharmaceutically acceptable salt or a pharmaceutical composition thereof, and such a MIEK inhibitor or a pharmaceutically acceptable salt or a pharmaceutical composition thereof, for use in treating a hematological cancer.

In a related aspect, the invention provides a kit containing a dose of a SOS1 inhibitor, or a pharmaceutically acceptable salt or a pharmaceutical composition thereof, and dose of a MEK inhibitor or a pharmaceutically acceptable salt or a pharmaceutical composition thereof, in an amount effective to inhibit proliferation of cancer cells, particularly SOS1 over-expressing cancer cells, in a subject. The kit in some cases includes an insert with instructions for administration of the a SOS1 inhibitor, or a pharmaceutically acceptable salt or a pharmaceutical composition thereof, and the MEK inhibitor or a pharmaceutically acceptable salt or a pharmaceutical composition thereof. The insert may provide a user with one set of instructions for using the a SOS1 inhibitor, or a pharmaceutically acceptable salt or a pharmaceutical composition thereof, in combination with the MEK inhibitor or a pharmaceutically acceptable salt or a pharmaceutical composition thereof.

The following Examples are intended to illustrate further certain embodiments of the invention and are not intended to limit the scope of the invention.

### Example A

### In Vivo Models for Examining SOS1 Inhibitor—MEK Inhibitor Combinations

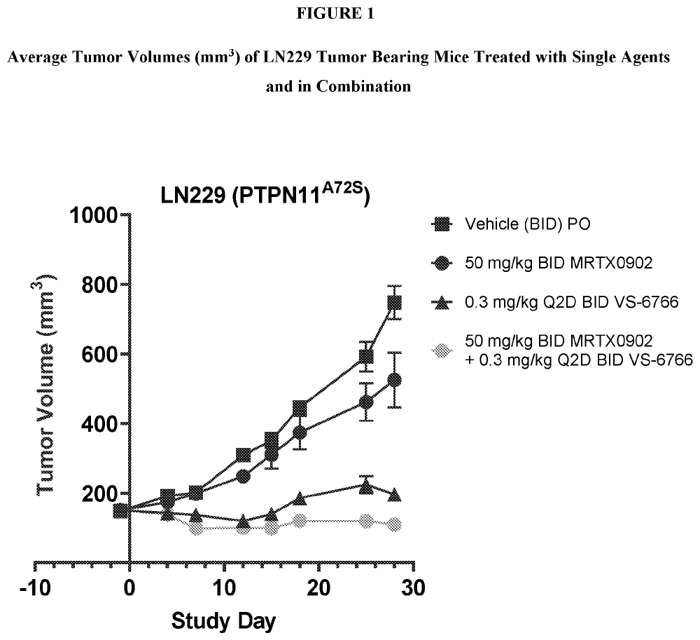

Immunocompromised nude/nude mice are inoculated in the right hind flank with LN229 cells harboring the A72S mutation. When tumor volumes reach approximately 150 mm3 in size, the mice are divided into four groups of 5 mice each. The first group is administered vehicle only. The second group is administered a single agent dose of the SOS1 inhibitor MRTX0902 at a 50 mg/kg twice daily (BID) concentration that yields a maximal biological effect but does not result in complete tumor regression. The third group is administered a single agent dose of the MEK inhibitor VS-6766 at 0.3 mg/kg twice daily (BID) as a single agent on Monday, Wednesday, and Friday (Q2D) concentration that yields less than maximal biological effect and does not result in complete tumor regression. The fourth group is administered the single agent dose of the SOS1 inhibitor in combination with the single agent dose of the MEK inhibitor. The treatment period was 28 days. Tumor volumes are measured using a caliper every two—three days and tumor volumes are calculated by the formula: 0.5×(Length×Width)₂. A greater degree of tumor growth inhibition for the combination in this model demonstrates that the combination therapy is likely to have a clinically meaningful benefit to treated subjects relative to treatment with only a MEK or SOS1 inhibitor.

[0153]20 nude/nude mice were inoculated in the right hind limb with 5×10⁶ LN229 cells. When tumor volume reached ˜150 mm3 (Study Day 0), 5 mice in each of the four groups were administered p.o. daily for 28 days: vehicle only (0.5% MC (4000 cps)/0.2% Tween80 in water), 50 mg/kg BID of the SOS1 inhibitor (R)-2-methyl-3-(1-((4-methyl-7-morpholinopyrido[3,4-d]pyridazin-1-yl)amino)ethyl)benzonitrile (MRTX0902) (0.5% MC (4000 cps)/0.2% Tween80 in water), 0.3 mg/kg BID Q2D of MEK inhibitor VS-6766 (5% DMSO, 10% HPCD in water), or 50 mg/kg BID MRTX0902 and 0.3 mg/kg BID Q2D. Tumor volumes, measured at pre-specified days, for the five mice per group were averaged and are reported for LN229 cells in Table 1 and FIG. 1.

TABLE 1
Average Tumor Volumes (mm³) of LN229 Tumor Bearing
Mice Treated with Single Agents and in Combination

| | | | VS-6766 | |
|---|---|---|---|---|
| | | | 0.3 mg/kg | |
| Study | | MRTX0902 50 | BID | MRTX0902 + |
| Day | Vehicle | mg/kg BID | MWF (Q2D) | VS-6766 |
| 0 | 149 | 151 | 151 | 152 |
| 4 | 192 | 174 | 143 | 138 |
| 7 | 202 | 199 | 137 | 99 |
| 12 | 309 | 248 | 120 | 101 |
| 15 | 352 | 310 | 140 | 99 |
| 18 | 446 | 374 | 186 | 120 |
| 25 | 592 | 462 | 225 | 119 |
| 28 | 748 | 525 | 196 | 110 |

As shown in Table 1 and FIG. 1, the administration of MRTX0902 at 50 mg/kg twice daily as a single agent resulted in 37.6% tumor growth inhibition at day 28. The administration of VS-6766 at 0.3 mg/kg twice daily as a single agent on Monday, Wednesday, and Friday resulted in 92.5% tumor growth inhibition at day 28. The combination of MRTX0902 at 50 mg/kg BID and VS-6766 at 0.3 mg/kg BID Q2D resulted in −27.6% tumor regression at day 28.

These results demonstrate that the combination therapy resulted in greater amount of tumor growth inhibition compared to either single agent alone demonstrating enhanced in vivo anti-tumor efficacy of the combination against PTPN11 A72S expressing cancer.

### Example B

### In Vivo Models for Examining SOS1 Inhibitor—MEK Inhibitor Combinations

Immunocompromised nude/nude mice are inoculated in the right hind flank with NCI-H1435 cells harboring NF1 K615N mutation. When tumor volumes reach approximately 150 mm3 in size, the mice are divided into four groups of 5 mice each. The first group is administered vehicle only. The second group is administered a single agent dose of the SOS1 inhibitor MRTX0902 at a 50 mg/kg twice daily (BID) concentration that yields a maximal biological effect but does not result in complete tumor regression. The third group is administered a single agent dose of the MEK inhibitor VS-6766 at 0.3 mg/kg twice daily (BID) as a single agent on Monday, Wednesday, and Friday (Q2D) concentration that yields less than maximal biological effect and does not result in complete tumor regression. The fourth group is administered a single agent dose of the SOS1 inhibitor in combination with a single agent dose of the MEK inhibitor. The treatment period was 27 days. Tumor volumes are measured using a caliper every two—three days and tumor volumes are calculated by the formula: 0.5×(Length×Width)₂. A greater degree of tumor growth inhibition for the combination in this model demonstrates that the combination therapy is likely to have a clinically meaningful benefit to treated subjects relative to treatment with only a MEK or SOS1 inhibitor.

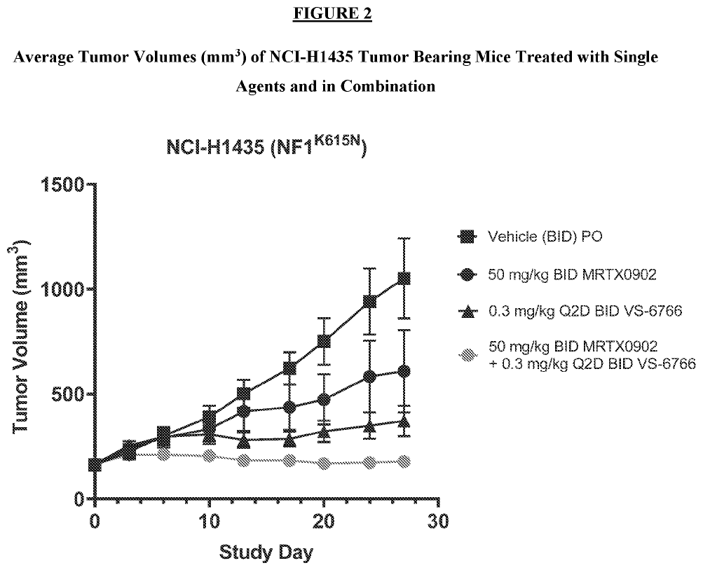

20 nude/nude mice were inoculated in the right hind limb with 5×10⁶ NCI-H1435 cells. When tumor volume reached ˜150 mm3 (Study Day 0), 5 mice in each of the four groups were administered p.o. daily for 27 days: vehicle only (0.5% MC (4000 cps)/0.2% Tween80 in water), 50 mg/kg BID of the SOS1 inhibitor (R)-2-methyl-3-(1-((4-methyl-7-morpholinopyrido[3,4-d]pyridazin-1-yl)amino)ethyl)benzonitrile (MRTX0902) (0.5% MC (4000 cps)/0.2% Tween80 in water), 0.3 mg/kg BID Q2D of MEK inhibitor VS-6766 (5% DMSO, 10% HPCD in water), or 50 mg/kg BID MRTX0902 and 0.3 mg/kg BID Q2D VS-6766. Tumor volumes, measured at pre-specified days, for the five mice per group were averaged and are reported for NCI-H1435 cells in Table 2 and FIG. 2.

TABLE 2
Average Tumor Volumes (mm³) of NCI-H1435 Tumor Bearing
Mice Treated with Single Agents and in Combination

| | | | VS-6766 | |
|---|---|---|---|---|
| | | | 0.3 mg/kg | |
| Study | | MRTX0902 50 | BID | MRTX0902 + |
| Day | Vehicle | mg/kg BID | MWF (Q2D) | VS-6766 |
| 0 | 161 | 164 | 166 | 167 |
| 3 | 225 | 241 | 254 | 210 |
| 6 | 300 | 292 | 296 | 211 |
| 10 | 390 | 333 | 307 | 205 |
| 13 | 501 | 417 | 281 | 183 |
| 17 | 622 | 437 | 286 | 182 |
| 20 | 750 | 474 | 322 | 169 |
| 24 | 941 | 583 | 349 | 173 |
| 27 | 1051 | 608 | 371 | 178 |

As shown in Table 2 and FIG. 2, the administration of MRTX0902 at 50 mg/kg twice daily as a single agent resulted in 49.8% tumor growth inhibition at day 27. The administration of VS-6766 at 0.3 mg/kg twice daily as a single agent on Monday, Wednesday, and Friday resulted in 76.4% tumor growth inhibition at day 27. The combination of MRTX0902 at 50 mg/kg twice daily and VS-6766 at 0.3 mg/kg twice daily on Monday, Wednesday, and Friday resulted in 98.1% tumor regression at day 27.

These results demonstrate that the combination therapy resulted in greater amount of tumor growth inhibition compared to either single agent alone demonstrating enhanced in vivo anti-tumor efficacy of the combination against NF1 K615N expressing cancer.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of treating cancer in a subject in need thereof, comprising administering to the subject a therapeutically effective amount of a combination of the SOS1 inhibitor: 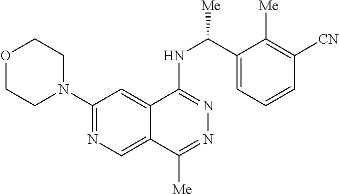 [SMILES: Cc1c(C#N)cccc1[C@@H](C)Nc1nnc(C)c2cnc(N3CCOCC3)cc12]
(R)-2-methyl-3-(1-((4-methyl-7-morpholinopyrido[3,4-d]pyridazin-1-yl)amino)ethyl)benzonitrile or a pharmaceutically acceptable salt thereof, and a MEK inhibitor.

2. The method according to claim 1, wherein the MEK inhibitor is selected from VS-6766, binimetinib, cobimetinib, selumetinib, trametinib, PD-325901, CI-1040 and CI-1040 or TAK733, or a pharmaceutically acceptable salt thereof.

3. The method according claim 1, wherein the MEK inhibitor is VS-6766.

4. The method according to claim 1, wherein the MEK inhibitor is binimetinib.

5. The method according to claim 1, wherein the MEK inhibitor is cobimetinib.

6. The method according to claim 1, wherein the MEK inhibitor is CI-1040 or TAK733.

7. The method according to claim 1, wherein the SOS1 inhibitor and the MEK inhibitor are administered on the same day.

8. The method according to claim 1, wherein the SOS1 inhibitor and the MEK inhibitor are administered on different days.

9. The method according to claim 1, wherein the SOS1 inhibitor is administered at a maximum tolerated dose.

10. The method according to claim 1, wherein the MEK inhibitor is administered at a maximum tolerated dose.

11. The method according to claim 1, wherein the SOS1 inhibitor and the MEK inhibitor are each administered at a maximum tolerated dose.

12. The method according to claim 1, wherein the MEK inhibitor is administered at below maximum tolerated dose.

13. The method according to claim 1, wherein the SOS1 inhibitor is administered at below maximum tolerated dose.

14. The method according to claim 1, wherein the SOS1 inhibitor and the MEK inhibitor are each administered at below maximum tolerated dose.

15. The method according to claim 1, wherein the therapeutically effective amount of the combination of the SOS1 inhibitor and the MEK inhibitor results in an increased duration of overall survival, an increased duration of progression free survival, an increase in tumor growth regression, an increase in tumor growth inhibition or an increased duration of stable disease in the subjects relative to treatment with only the SOS1 inhibitor.

16. The method according to claim 1, wherein the therapeutically effective amount of the combination of the SOS1 inhibitor and the MEK inhibitor results in an increased duration of overall survival, an increased duration of progression free survival, an increase in tumor growth regression, an increase in tumor growth inhibition or an increased duration of stable disease in the subjects relative to treatment with only the MEK inhibitor.

17. A pharmaceutical composition, comprising a therapeutically effective amount of a combination of a SOS1 inhibitor and a MEK inhibitor according to claim 1, and a pharmaceutically acceptable excipient.

18. A method of method for inhibiting SOS1 activity in a cell, comprising contacting the cell in which inhibition of SOS1 activity is desired with an effective amount of a combination of the SOS1 inhibitor: 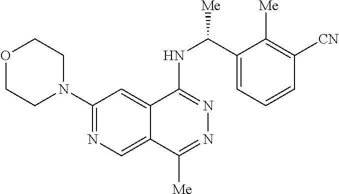 [SMILES: Cc1c(C#N)cccc1[C@@H](C)Nc1nnc(C)c2cnc(N3CCOCC3)cc12]
(R)-2-methyl-3-(1-((4-methyl-7-morpholinopyrido[3,4-d]pyridazin-1-yl)amino)ethyl)benzonitrile or a pharmaceutically acceptable salt thereof, and a MEK inhibitor.

19. The method of claim 18, wherein the MEK inhibitor is selected from VS-6766, binimetinib, cobimetinib, selumetinib, trametinib, PD-325901, CI-1040 and TAK733, or a pharmaceutically acceptable salt thereof.

20. The method according to claim 18, wherein the MEK inhibitor is VS-6766.

21. The method according to claim 18, wherein the MEK inhibitor is binimetinib.

22. The method according to claim 18, wherein the MEK inhibitor is cobimetinib.

23. The method according to claim 18, wherein the MEK inhibitor is CI-1040 or TAK733.

24. The method according to claim 1, wherein the MEK inhibitor synergistically increases the sensitivity of cancer cells to the SOS1 inhibitor.

25. A method for increasing the sensitivity of a cancer cell to a SOS1 inhibitor comprising administering to a subject undergoing treatment with an effective amount of a combination the SOS1 inhibitor: 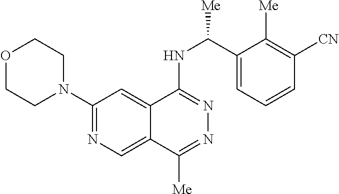 [SMILES: Cc1c(C#N)cccc1[C@@H](C)Nc1nnc(C)c2cnc(N3CCOCC3)cc12]
or a pharmaceutically acceptable salt thereof, and a MEK inhibitor, wherein the MEK inhibitor synergistically increases the sensitivity of the cancer cell to the SOS1 inhibitor.

26. The method according to claim 1, wherein the therapeutically effective amount of the SOS1 inhibitor in the combination is between about 0.01 to 100 mg/kg per day.

27. The method according to claim 25, wherein the therapeutically effective amount of the SOS1 inhibitor in the combination is between about 0.1 to 50 mg/kg per day.

28. The method according to claim 1, wherein the therapeutically effective amount of the MEK inhibitor in the combination is between about 0.01 to 100 mg/kg per day.

29. The method of claim 27, wherein the therapeutically effective amount of the MEK inhibitor in the combination is between about 0.1 to 50 mg/kg per day.

30. The method according to claim 1, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

31. The method of claim 29, wherein the cancer is a SOS1-associated cancer.

32. The method of claim 29, wherein the cancer is a KRas G12C-associated cancer.

33. The method of claim 30, wherein the cancer is non-small cell lung cancer.

34. A kit comprising the pharmaceutical composition of claim 17 for treating SOS1-associated cancer in a subject.

35. The kit according to claim 34, further comprising an insert with instructions for administration of the pharmaceutical composition(s).
